(19) United States
(12) Patent Application Publication
(10) Pub. No.: US 2017/0107709 A1
(43) Pub. Date: Apr. 20, 2017

(54) TOILET ODOR ELIMINATION DEVICE

(71) Applicants: Marvin L. Kausch, Ormond Beach, FL (US); Marilyn Seuss, Ormond Beach, FL (US)

(72) Inventors: Marvin L. Kausch, Ormond Beach, FL (US); Marilyn Seuss, Ormond Beach, FL (US)

(21) Appl. No.: 15/295,371

(22) Filed: Oct. 17, 2016

Related U.S. Application Data
(60) Provisional application No. 62/243,541, filed on Oct. 19, 2015.

(51) Int. Cl. E03D 9/052; B01D 53/04; A61L 9/16; B01D 53/58; B01D 53/82; A47K 13/30; B01D 46/00

(52) U.S. Cl. CPC E03D 9/052; A47K 13/307; B01D 53/04; B01D 46/0038; B01D 46/0036; B01D 53/58; B01D 53/82; A61L 9/16; B01D 2251/306; B01D 2253/102; B01D 2257/406; A61L 2209/14; A61L 2209/11

(57) ABSTRACT

A device for minimizing escape of malodorous gases from a toilet into the surrounding environment. In particular, a device for removing toilet-related odors, comprising a manifold, air-blower in fluid communication with intake and exhaust ports, that creates a horizontal flow of recirculating air at the top of the toilet bowl. The recirculating air passes through a filter that contains activated carbon and a potassium-permanganate impregnated zeolite material.

### CROSS-REFERENCE TO RELATED APPLICATION

The present application claims priority to U.S. Provisional Patent Application No. 62/243,541 filed on Oct. 19, 2015, entitled “TOILET ODOR ELIMINATION DEVICE” the entire disclosure of which is incorporated by reference herein.

### BACKGROUND OF THE INVENTION

Technical Field

The technology described herein generally relates to toilets, and more particularly relates to devices for minimizing escape of malodorous gases from a toilet into the surrounding environment.

Description of Related Art

Many have long endeavored to mitigate the spread of unpleasant odors related to the normal physiological function of the human digestion and elimination system. Such odors can be unpleasant in a toilet or bathroom location, may even contaminate the fresh air of adjacent areas, and can travel from the immediate locale of the toilet by permeating and clinging to a person's clothes. Furthermore, gastrointestinal diseases and digestive conditions typically give rise to excessively putrid odors which are more pungent than those produced by the majority of people. For people suffering from those afflictions, their odors can overwhelm a bathroom and surrounding area, and are thereby incompatible with comfortable habitation.

According to the Nov. 13, 2014 report, “Digestive Diseases Statistics for the United States”, from the National Institute of Health (http://www.niddk.nih.gov/health-information/health-statistics/Pages/digestive-diseases-statistics-for-the-united-states.aspx) digestive diseases are prevalent in 60 to 70 million people in the United States alone.

RightDiagnosis.com lists over 200 individual diagnoses in which foul intestinal odor and or stool can be considered a symptom. A non-exhaustive list of digestive diseases, syndromes and related issues includes: gluten intolerance, lactose intolerance, diets high in fiber, cruciferous vegetables or beans, chronic constipation, diverticular disease, irritable bowel syndrome, Crohn's disease, gall bladder disease, pancreatic disease, Celiac's disease, gastrointestinal infections such as c-diff or food-borne illnesses, colitis, liver disease, short bowel syndrome, and colon cancer.

In addition to the aforementioned 60-70 million people in the USA, some of the worst odor problems are caused by weight loss surgeries such as the roux en y gastric bypass (RNY), the biliopancreatic diversion with duodenal switch (DS) and the more recent stomach intestinal pylorus-sparing surgery (SIPS). Over 2.5 million people in the USA have already had a weight-loss surgery and there are approximately 250,000 more procedures performed each year. These surgeries allow incompletely digested fats and amino acids into the large colon where they ferment causing large volumes of intestinal gas. The surgically forced incomplete digestion creates a markedly increased amount of stool. Both of these factors are implicated in the horrendous bathroom odors associated with mal-absorptive weight loss surgeries. Other side-effects such as bloating, flatulence, diarrhea, constipation, and incontinence only compound the problem. While not all of those afflicted will experience these symptoms all the time, even an infrequent occurrence can be extremely disruptive. These odors thus cause an especially sorrowful problem for those who are entering into a new post-morbidly obese lifestyle: rather than beginning to enjoy life as a “normal” weight person, they find that their entrance into a new social life is stymied by horrifying personal odors that is often so severely embarrassing that it can lead to social isolation. This, in turn, translates into missed family and social gatherings, spoiled intimate encounters, as well as missed professional meetings, lost business opportunities, and may also contribute to work absenteeism. In one recent example, an individual who “struggled with extreme gas and uncontrollable diarrhea” following gastric bypass surgery, was terminated from his employment because of the effect on “customer relations” (see, nypost.com/2015/10/02/pork-roll-company-fired-my-hubby-for-farting-wife-says/).

Exhaust fans in toilet rooms can take 10 to 30 minutes or more, depending on the size of the room and the number of toilets, to clear the ambient air in a room of foul or putrid odors. In high use toilet rooms, the exhaust fans may be inadequate to ever clear the odor from the room. This can cause an insurmountable problem of odor removal in smaller spaces such as toilets in commercial locations, small offices such as doctor or dentist offices, spas, schools, conference facilities, buses, trains, etc. Inadequate exhaust systems in high volume toilet rooms can create a critical problem in large venues, such as airports, sports events, concerts, and restaurants, where food and dining may be occurring nearby. For example, in one instance, a commercial airliner was forced to make an emergency landing due to a passenger attempting to conceal their flatulence by lighting matches onboard. (See usatoday30.usatoday.com/travel/flights/2006-12-05-flatulence-landing_x.htm.)

Furthermore, exhaust fans actually tend to pull toilet odors (that may otherwise be localized near the ground or toilet bowl) more fully into the room, arguably making the problem worse before it gets better as the whole volume of air is recycled. This makes it more likely that odors may permeate and cling to a person's clothing so that they leave the toilet room in a cloud of foul odor and carry it on their body until it dissipates.

The occurrence of the odors is not limited to toilets and bathrooms but, in the case of flatulence, can be manifested in such situations as public transportation (in confined spaces on airline travel, trains, buses, boats), open plan workspaces (such as “cube farms”), other office environments, schools, classrooms, clinics, hotels, conference facilities, courtrooms, restaurants, and malls, not to mention during social events, one on one meetings, dates, meals out, and even intimate encounters.

Therefore, putrid odors from intestinal gas and stool present an embarrassing problem, not only to tens of millions of people directly suffering from the aforementioned issues, but also the tens of millions who are impacted indirectly by the virtue of being in close physical proximity to the affected: family, friends, as well as the general public.

By 2030 it is projected that there will be more than 72 million people in the U.S of the age 65 and older. (U.S. Census Bureau https://www.census.gov/prod/2010pubs/p25-1138.pdf) Age is a major risk factor for increased incidence of gastrointestinal disease and, on top of being at increased risk for gastrointestinal disease due to increased age, certain physiological changes occur with aging that lead to further symptoms of gastrointestinal dysfunction in the elderly (e.g., dysphagia, irritable bowel syndrome, primary constipation, mal-digestion and medication side effects).

Concomitant with the aging of the population will be the increased occurrence of foul odor symptoms as well as potentially less control of them, leading to more episodes that result in embarrassing or unacceptable odor problems. There will be an increased demand for methods of keeping geriatric communities odor free. Odor control is not only an issue of quality of life for long-term care residents, but it is also an issue of first impressions for residential homes, retirement communities, assisted living facilities, memory loss units and skilled nursing homes. What visitors to a nursing home can perceive with their noses might very well be the first impression they get. A bad first impression due to malodors from toileting residents could easily translate into an economic impact in that market.

Devices in the prior art have generally not been effective. One reason is that many use “single pass” operation, which is generally not sufficient to deal with the volume of odors. Some devices of the prior art manage the air that is removed from the toilet bowl as follows. In some implementations, the air is exhausted through a hose to a location outside of the toilet room (e.g., U.S. Pat. No. 6,550,072), which may not be helpful to those on the outside, or may be difficult to implement: for example, where a toilet abuts an exterior wall, one could drill or cut a hole in the wall near to the toilet and insert a vent hose through the hole directly to the outside. In other situations, it has been suggested to run lengths of (possibly unsightly) hosing from the toilet, up the wall, and over to a ceiling exhaust fan. In more extreme situations, it may prove necessary to cut a hole in one or more walls in order to vent to a separate location. In other variants, the air is run through a filter ONCE, and then exhausted into the toilet room atmosphere (e.g., U.S. Pat. No. 8,337,602), which is generally not adequate. In still other variants, it is exhausted through the plumbing of the toilet, e.g., via an overflow pipe or P-trap (e.g., U.S. Pat. Nos. 7,117,584, and 5,386,594), which is complicated to plumb. Another way in which the devices of the prior art have not been effective is that the filter materials used are inadequate to scrub the air of many or most of the most dislikable components of toilet odors.

Accordingly, there is a need for a device that will remove all odors from the use of toilets, including the most pungent odors that can be produced by persons suffering from digestive tract ailments or who have had weight-loss surgery.

The discussion of the background herein is included to explain the context of the technology. This is not to be taken as an admission that any of the material referred to was published, known, or part of the common general knowledge as at the priority date of any of the claims found appended hereto.

Throughout the description and claims of the instant application the word “comprise” and variations thereof, such as “comprising” and “comprises”, is not intended to exclude other additives, components, integers or steps.

### SUMMARY OF THE INVENTION

The disclosure comprises an apparatus for removing odors from a toilet environment, the apparatus comprising: a manifold disposed on top of the toilet bowl, wherein the manifold is configured to create a circuit of closed air; and a filter in fluid communication with the manifold, through which odorous air passes, wherein the filter comprises activated carbon and potassium permanganate.

The instant disclosure includes a method of removing odors from a toilet environment, the method comprising: creating a circuit of closed air from the toilet bowl to an attached manifold; and passing the closed air over a filter in fluid communication with the manifold, wherein the filter comprises activated carbon and potassium permanganate.

### BRIEF DESCRIPTION OF THE DRAWINGS

FIGS. 1A and 1B are a schematic diagram, in two views, of an embodiment of the invention: the upper panel shows a view from above, and the lower panel shows a view from the side. A toilet bowl 5, a toilet seat 10, one or more air tight seals/gaskets 15 (disposed between the device and the toilet, the device and the toilet seat, and the toilet seat and the lid, according to the embodiment), an airflow manifold 20, which contains an air exhaust port 30, and air intake port 40, and creates horizontal air flow 50 between the intake port and exhaust port. A canister 60 is connected to the air exhaust port, and contains material which filters the air, and airflow movement is enabled by a blower or fan 70, which is connected to canister 60 via canister-to-blower adapter 80.

The embodiment of FIGS. 1A and 1B is one in which seat 10 is a separate item from the manifold 20. In another embodiment, seat 10 and manifold 20 are moulded into a single unit.

FIG. 2 shows a perspective view of the device of FIGS. 1A and 1B, fitted to a toilet. An air tight gasket 15 is visible on the lower side of the toilet lid.

In the embodiment of FIGS. 1A-2, filter cartridge 60 is connected to manifold 20, so that the items form a self-contained unit. In an alternate embodiment, shown in with FIG. 7, the filter unit 150 is located separately from the manifold.

FIG. 3 shows a removable toilet seat 10 compatible with the manifold 20 shown in FIGS. 1A and 1B. In this embodiment, the manifold is mounted on the toilet between an existing seat and the toilet bowl rim. In another embodiment, a combined manifold/toilet seat unit is installed in place of an existing toilet seat.

FIG. 4 shows the device and toilet seat in the raised position. The location of the seal/gasket 15 on the bottom of the manifold 20, is as shown in FIGS. 1A and 1B. FIG. 4 also shows the location of the filter canister when the seat is in the up position. The location of the canister, just to the side and rear of the toilet, facilitates the two positions of the seat (“up” and “down”) in that it does not obstruct either, and therefore the device is compatible with routine use of a toilet by both men and women. In either embodiment, whether the manifold and seat are combined or separate, it is possible to raise the seat and manifold in this manner.

FIG. 5 shows a side view of an embodiment of the filter canister that comprises air tube 100, housing 110, three fiber material separators 120, activated carbon filter material 130, and zeolite permeated with potassium permanganate filter material 140.

FIG. 6 shows a perspective view of the top of the filter canister 60, revealing housing 110 having inlet 62 in tube 100, and outlet 64 of the filter material. Locking tab 66 permits safe and secure closure of the canister after replenishment of filter material. Clamp 67 secures air tube 100 to the housing.

FIG. 7 shows a toilet seat 10 mounted to a manifold 20 allowing the horizontal air flow previously described for use with an external filter unit 150 (FIG. 8). The external filter unit connects to manifold 20 via tubing 152 for outbound air flow and 154 for inbound air flow. External filter unit 150 is not mounted on the toilet but can be installed in a remote location, such as on the floor of the bathroom, or mounted inside or on the wall behind the toilet. An interior mount (such as between two wall studs, obscured by drywall) is more discrete. An alternate embodiment may require construction of a tailored niche or recessed portion of the wall to accommodate the filter unit. In still other embodiments, the filter unit could be installed in, for example, an under-sink vanity cabinet adjacent to the toilet.

FIG. 8 shows the internal construction of an embodiment of the external filter cartridge 150. The external filter unit 150 contains blower 70, separators 120, filter materials 130 and 140.

FIGS. 9-11 show multiple exemplary AC-to-DC wall adapters 160 with sufficient wattage to operate the blower. An alternate power source would be a 12 V DC battery 170.

FIG. 12 is an exemplary schematic of the controller. A push-button switch 210, when pressed, starts the blower operation for a time, t, which could be 5 minutes, or more or less. Operation of the device, on/off, may be over-ridden by the consumer by further pressing switch 210. The circuit further consists of the power source, 160 or 170, a voltage regulator 200 to drop the battery voltage to a suitable level for the microcontroller supply VDD, a microcontroller 220, running a program such as described in FIG. 11. The microcontroller output drives a power transistor 230, either bipolar or FET which controls motor on-off current.

FIG. 13 shows an exemplary flow-chart for operation of software that controls the micro-controller of FIG. 10.

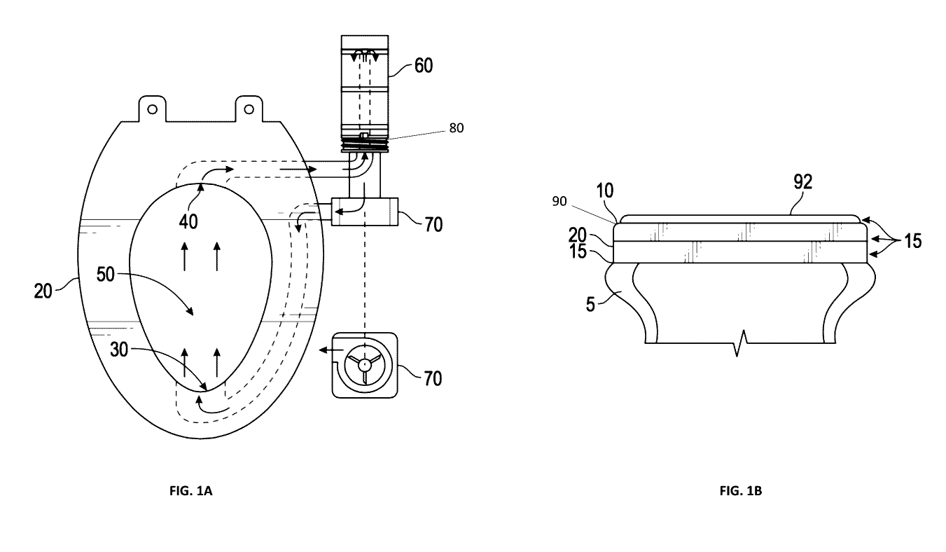

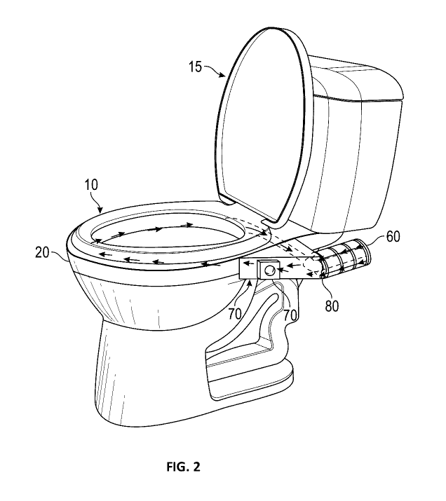

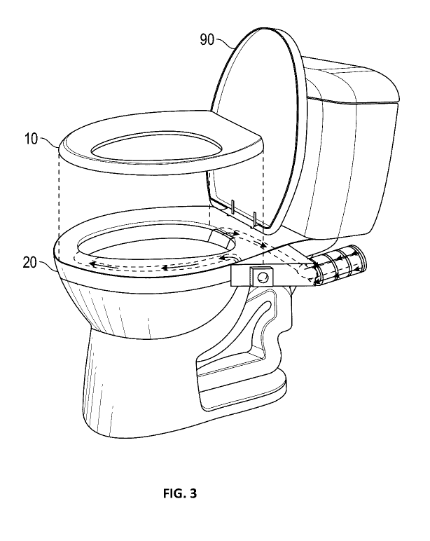

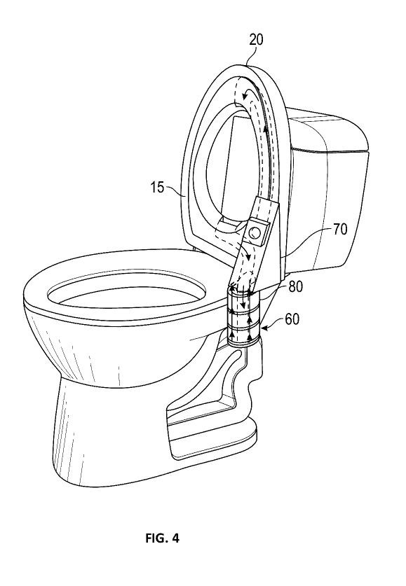

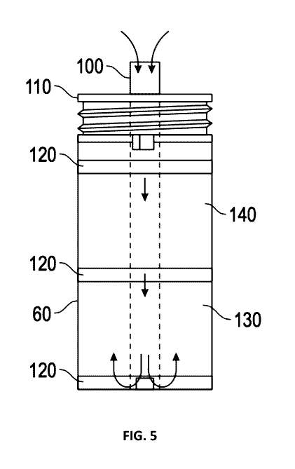

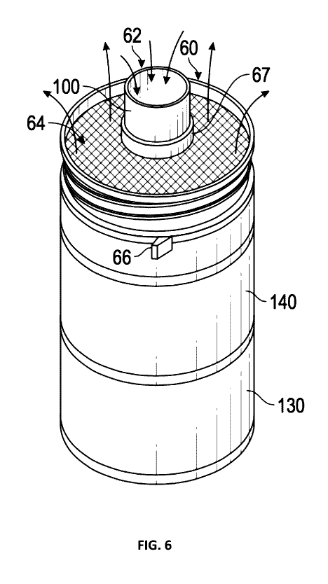

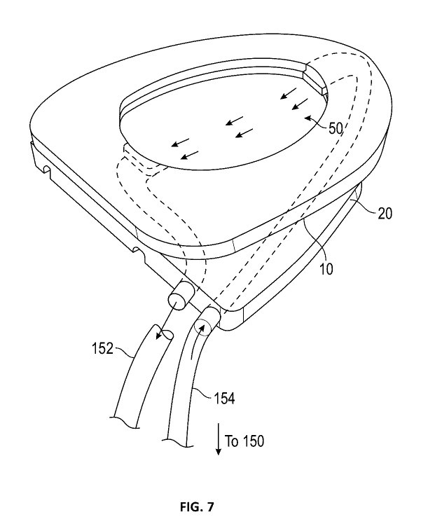

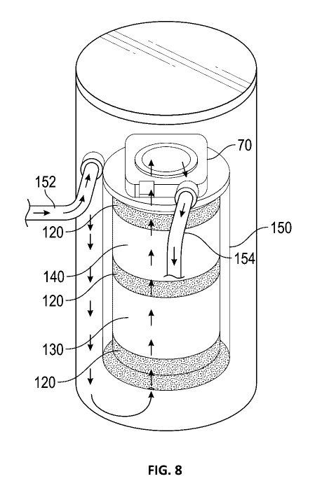

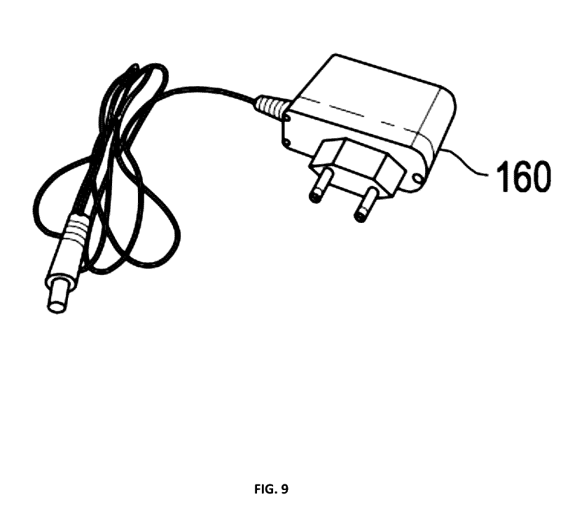

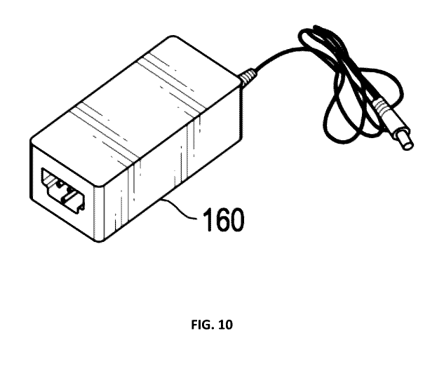

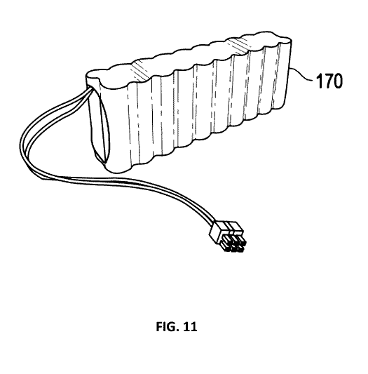

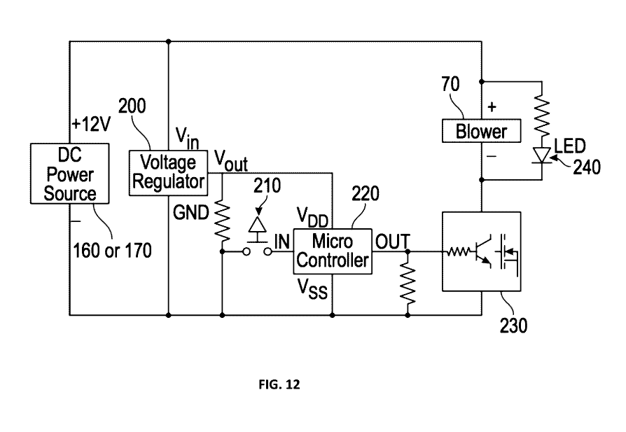

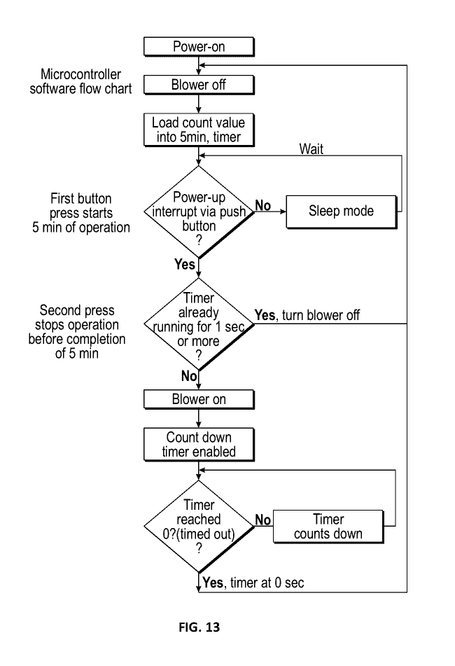

Like reference symbols in the various drawings indicate like elements.

### DETAILED DESCRIPTION OF PREFERRED EMBODIMENTS

### Overview

The instant technology is directed to devices for minimizing escape of malodorous gases from an individual, such as when using a toilet, into the surrounding environment.

Embodiments of the invention contain the following features: fully enclosed and sealed airflow; continuously circulating air flow; filtered air is reintroduced back into the toilet bowl, not into the room atmosphere, thereby ensuring minimal to no escape of odors to outside the toilet bowl.

Additionally, the device may include such user interface features as an on/off switch, time delay shut off feature, an indicator to demonstrate when the filter(s) need to be changed, and a power on indicator light.

The filter uses a chemically activated molecular sieve to remove odors and does not cover the odor with any scent. This is an important feature because, increasingly, clinics, schools, and workplaces are implementing fragrance free policies due to an uptick in asthma, COPD, and allergies. (See, e.g., www.fragrancex.com/fragrance-information/fragrance-industry.htm.)

The device differs from prior art in at least the following respects.

First, it utilizes closed-circulated air, by creating a sealed toilet bowl that recirculates the enclosed air volume.

Second, many devices in the prior art add a fragrance to the air, whereas the present device does not.

Prior art filters use activated carbon as a single filter medium. The device of the present invention additionally includes a potassium permanganate impregnated zeolite which ensures removal of the most obnoxious components of toilet odor.

The device of the present invention pivots the filter canister into an easy to access position when changing the filter, an aspect that also allows for easier cleaning of the device and the toilet bowl, as well as consecutive use of the toilet by both males and females.

The device of the present invention needs no exhaust to the outside of the bathroom, such as through the wall, but instead is self-contained.

Embodiments of the device of the present invention utilizes a single unit construction: only two standard mounting screws secure the manifold of the unit to the toilet.

### Characteristics of the Invention

The principal components of a device of the invention include:
1. An air-flow manifold. The manifold comprises air intake and exhaust ports to create an air flow 50 from front to rear of manifold/seat. In one embodiment, the manifold is integrated into the toilet seat as one piece. In a second embodiment, the toilet seat is a separate component from the manifold.
2. Air sealing gaskets between the manifold and toilet bowl, and between manifold and toilet seat if the manifold is not integrated into the toilet seat, and between toilet seat and lid.
3. A blower or fan, operating under 12 V DC or some other convenient voltage supplied via a battery, or via the electrical mains through an AC to DC converter.
4. A molecular odor scrubbing air filter unit. This may be a stand-alone unit with hose connections to the manifold, or may be an integrated replaceable cartridge design. Both embodiments allow for renewal or replacement of exhausted filter materials. In the circumstance of a portable embodiment of the invention, this filter could be a hand-held cartridge.
5. A source of power for the blower such as a wall mounted low voltage transformer, or a DC battery, and a control of the power source such as with an on-off switch. In some embodiments, the toilet is fitted with a sensor that determines, e.g., when a person has seated themselves on the toilet or has raised the toilet lid, and then automatically activates the blower.

Embodiments of the invention are now described in conjunction with the figures.

Toilet bowl 5, seat 10, and lid 92, constitute a sealed enclosure that minimizes leak of bowl vapors into the surrounding room. A unique feature of the invention is that the recycling internal airflow does not vent to the bathroom.

An air flow manifold 20 is configured to fit between a standard toilet seat and the top of the toilet bowl. Its design may include gaskets 15 or sealing material to ensure that no bowl vapors escape to the side. In one embodiment, the manifold may be integrated into the toilet seat 10. The intake 40 is contained within the manifold, directing air from the toilet bowl into the ductwork of the manifold 20, then into filter unit 60, then through the blower 70, then back out of the blower into the manifold 20, and finally out of the exhaust port 30 where it is reintroduced into the toilet, thereby creating a continuous recirculation of air in the toilet bowl. Thus, in a preferred embodiment, the blower sucks air from the toilet, and through the filter.

Air flow 50 in the toilet bowl, created by the device, forms a horizontal layer, in the plane of the manifold and parallel with the top of the toilet bowl. The air is directed to flow from the exhaust port 30 at inside front edge of the seat to a rear mounted intake port 40. Although embodiments having other directions of air flow can be constructed (e.g., where 30 is at the rear, and 40 is at the front, or embodiments in which the airflow is from side-to-side also) the front to rear direction of flow is most hygienic and therefore most effective.

A blower 70 causes air to flow from the intake port 40 (mounted at the rear of the toilet in the embodiment shown), through filter unit 60, through the blower inlet 30. In various embodiments, the fan does not make an intrusive sound when operating and preferably is barely audible. In one embodiment, the blower is a commercially available NIDEC gamma-30 fan, though it would be understood that many different types of readily available fan may be used to construct an embodiment of the invention.

Air flow 50 continuously recirculates from the exhaust port 30 at the front of the toilet towards and into the rear toilet seat vent 40, then into the filter unit 60, then out of the filter through the blower 70, then is ducted to the front exhaust port vent 30. Air flow 50 acts to create a horizontal barrier to the vapors escaping from the toilet bowl. Malodorous gases in the toilet bowl, which could otherwise rise into the ambient room air, are swept into the horizontal flow of air created in the toilet bowl and directed through a molecular sieve in the filter unit 60 to scrub the odor molecules from the air.

After the user has completed a toilet session, the lid 92 may be closed so that the gasket/seal 90 confines the odors to the toilet bowl. In one embodiment, a magnetic latch (not shown in the FIGs.) ensures the air tight seal is maintained against the pressure impulse created by influx of water into toilet bowl during a flush. Together, these features prevent odorous vapors from escaping into the surrounding space, such as the cubicle or bathroom. While the user is seated on the toilet, the horizontal air flow continuously draws odorous air into the filter. If the user gets up and closes the lid, then the lid, gasket and toilet seat, in combination, contain the odors as the airflow draws them through the filter.

In another embodiment, the blower could be set to be in operation for a certain length of time, which is preferably be longer than the period of time for which the toilet is actually in use, to save the user from having to remember to switch the unit off. The horizontal air flow helps to prevent odors coming into the room. The air flow may be laminar or turbulent in nature. Flushing the toilet disrupts the airflow, so flushing when the lid is down is best, and maintaining the lid in the closed position even after the toilet is flushed is preferred. This is because a person's body can also act as a partial seal. When a person is not seated on the toilet, there is necessarily a greater level of exposure of the odors to the room.

There are at least three ways in which the device is designed to prevent odors from escaping during the flush operation. A first way is the airtight seals or gaskets that prevent any impulse of air caused by a flush from escaping under or around the toilet seat, any connections, the filter, or the blower.

A second way is the proper positioning of the user's body to occlude any air leakage from the toilet bowl during use. The user's body can also be used to seal the toilet during the actual flush. Often those who cause significant toilet odor problems will flush multiple times during one use. The term “courtesy flush” has been coined for that behavior (see, e.g., www.urbandictionary.com/define.php?term=courtesy+flush, and science.howstuffworks.com/science-vs-myth/everyday-myths/courtesy-flushes-useful-or-healthy.htm).

A third method to prevent odor escaping during the flush is for the toilet user to rise from the toilet seat, allowing the horizontal airflow to contain all odors for the amount of time it takes to close the airtight lid. Once the lid is closed, the gasket and magnetic latch maintain the airtight seal during the flush.

Air tight seals securely contain odorous vapors inside the toilet bowl, and the filter material scrubs the odiferous molecules from the air via a molecular sieve prior to the air being recirculated. Recirculation ensures that the air is repeatedly scrubbed to ensure the air is thoroughly cleaned of all odors.

The recirculation of the same toilet bowl vapors, ensures that there is no escape into ambient room atmosphere. This feature is especially important for persons who, due to medical conditions or mal-absorptive conditions, create especially voluminous amounts of gas and/or stool, or create especially putrid odors due to digestive mal-absorption and medical diseases. In these cases, a simple one-pass air flow, such as described in the prior art, is totally inadequate.

The filter is preferably provided as an easily replaceable cartridge design. The cartridge can be fitted with a lock 66 to force it into a position that creates an airtight seal. The lock 66 also prevents inadvertent loosening or access to the filter material by children or pets. The lock may comprise a simple pair of complementary surfaces that fit together tightly and require operating a latch or application of significant force to separate them.

In some embodiments, the filter material is provided in a cartridge form that is a replaceable insert that can be placed inside the canister (similar to the interchange of a HEPA-filter in a vacuum cleaner). In other embodiments, the canister and filter material inside are a self-contained unit that are replaced in their entirety (analogously to the cartridges in some types of under-sink water filter units).

The filter comprises proportions of activated carbon and potassium permanganate (KMnO4)-activated zeolite, and other filter/filler materials such as batting, paper, fabric, or wire mesh. These latter materials can separate the activated carbon from the zeolite, and can also be used as a packing to make sure that the filter material remains tightly and evenly distributed through the cartridge or canister. It is preferable for there to be a small space at end of canister to facilitate travel of the recirculating air back. The air intake tube 100 goes through the center of the filter material in the canister.

Activated carbon (sometimes referred to as activated charcoal) has been the filter material of choice in toilet odor filters known in the art. Sometimes, in the devices of the prior art, activated carbon is accompanied by a fragrance, which is utilized to conceal any odors that the activated carbon portion of the filter failed to remove. However, in most instances, a fragrance is not sufficient to mask the most unpleasant odors associated with toilet usage. In the present invention, both the specific filter materials and the order in which they are used in the filter system, are important to the overall success in odor removal, and ensure that no additional fragrance is required. In a preferred embodiment, the two filter materials are placed in series in the canister so that air passes through one first, then a second filter. It is preferable for the air from the toilet to pass through the activated carbon before it passes over the zeolite.

The odors caused by fecal material and gaseous excretions are best handled by a combination of potassium permanganate impregnated zeolite, and activated carbon.

The odorous compounds that the filter needs to retain, adsorb, or neutralize, include but are not limited to: indole, skatole, aldehydes and/or ketones (compounds that contain a carbonyl group), amines and other nitrogen containing compounds.

Indole, skatole, and ketones are best adsorbed by activated carbon.

Amines and other nitrogen containing compounds are not well adsorbed by activated carbon and benefit from the oxidative properties of potassium permanganate to be mitigated. A zeolite that has been impregnated with potassium permanganate oxidizes the amine and other nitrogen-containing compounds, and holds the byproduct (oxidized) compounds in the pore structure of the zeolite.

Aldehydes are quite easily oxidized by the potassium permanganate, whereas ketones are oxidized only with difficulty (and so are typically captured by the activated carbon). Aldehydes comprise low molecular weight compounds like formaldehyde and acetaldehyde, and higher molecular weight compounds such as benzaldehyde and toluene-aldehyde. Standard activated carbon will do a good job of adsorbing the larger aldehyde molecules, (benzaldehyde and toluene-aldehyde), but the smaller aldehyde molecules (such as formaldehyde and acetaldehyde) are not adsorbed well. The smaller molecules are typically oxidized.

The relative humidity found in bathroom environments can be quite high, and even greater than 60%. When the activated carbon is subjected to this high humidity, its pore structure can become filled with water, and the material significantly loses reactivity and adsorption capacity. With increasing humidity, the activated carbon will become less effective in removing pollutants, and in some rare cases will discharge pollutants when humidity and temperature are raised significantly and or quickly. This is not the case with the potassium permanganate impregnated media, as it is most effective in a humid environment.

Because of this it is best to use a dual stage filtration process: in a preferred embodiment, a two-stage filter system in which running the air through activated carbon first achieves a partial cleaning of the air before it passes through the potassium permanganate impregnated zeolite, which is positioned second in line in the airflow. That way, it will be possible to prolong the life of the potassium permanganate. In other embodiments, a blended mixture of potassium permanganate impregnated zeolite and activated carbon (or a potassium permanganate impregnated activated carbon) may be used, but it may not be as efficient as 2-stage filtration.

In other, less preferred embodiments, air passes over the potassium permanganate-activated zeolite before the activated carbon.

Zeolites come in many different particle sizes (as does activated carbon)—according to what size mesh the particles will fit through. Examples of suppliers of zeolite materials for the filter of the present invention include: Envirologek. “potassium permanganate activated zeolite EL600” (see, e.g., http://envirologek.com/shop/environmental-remediation-products/water-treatment-systems/potassium-permanganate-impregnated-zeolite-el600/). Another commercially available material is Hydrofill-600, blend of KMnO4 zeolite and carbon, which could be used in a single-stage filter.

In a preferred embodiment, the mesh size for both the activated carbon and the potassium permanganate impregnated zeolite is ¼-⅙ inch diameter. This mesh size enables minimally restricted air flow through the filter media. By using different fans and/or fan motors a ⅙-⅛ inch diameter or smaller mesh would also be effective. The finest mesh (sand size) option of 1/28- 1/48 inch diameter offers the highest surface area per unit of volume of media, and is desirable because the smaller volume of media that would then be effective would lead to a smaller sized filter unit. See www.bearriverzeolite.com/brz_particle_sizes.htm for exemplary zeolite categories. For small particle sizes, it is preferable to mix it with fibers, paper, etc., so as not to impede air-flow. In some embodiments in which small particle sizes are used, the filter material can be coated with fine sized particles of activated carbon and potassium permanganate zeolite. The particles may be bonded to the filter material in a manner understood by one of skill in the art.

In a preferred embodiment it is possible to take advantage of the fact that the very purple color of the potassium permanganate impregnated zeolite will slowly turn to brown as oxidation occurs, to create a visual indicator of filter exhaustion. In some embodiments, a small clear view window in the filter housing (not shown in the figures) permits a consumer or facility maintenance staff to monitor the color change over time and usage.

Air flow recirculation draws odor molecules through the molecular sieve of the filter material multiple times, each time trapping more odor, as compared to a single-pass filter of prior art. As the filter material loses effectiveness with use, this multiple pass design improves filter effectiveness. This action is similar to a swimming pool filter: continuous water flow filtering action catches contaminants missed by only a single pass design.

The device is easily installed on most toilets, for example by simply unscrewing the two bolts that attach a standard toilet seat to a standard toilet bowl, and replacing the original toilet seat with the device. Where the device is an integrated manifold and seat, it replaces the removed seat. Where the device is a manifold designed to reside between seat and bowl, the original seat is re-attached after installation of the manifold.

The filter location does not interfere with the normal down and up positions of a toilet seat in either an embodiment where it is connected directly to the manifold or in an embodiment in which it is externally situated. Additionally, in some embodiments, the filter cartridge rotates between horizontal and vertical positions as the seat is raised and lowered. When the seat is down, the filter cartridge is horizontally oriented toward the rear of the toilet where it is not likely to interfere with toilet use. When the toilet seat is raised, the filter cartridge rotates into a vertical position, bringing it into view, also allowing for easy access and replacement.

All references cited herein are incorporated by reference in their entireties.

The foregoing description is intended to illustrate various aspects of the instant technology. It is not intended that the examples presented herein limit the scope of the appended claims. The invention now being fully described, it will be apparent to one of ordinary skill in the art that many changes and modifications can be made thereto without departing from the spirit or scope of the appended claims.

What is claimed:

1. An apparatus for removing odors from a toilet environment, the apparatus comprising:
a manifold disposed on top of the toilet bowl, wherein the manifold is configured to create a circuit of closed air; and
a filter in fluid communication with the manifold, through which odorous air passes, wherein the filter comprises activated carbon and potassium permanganate.

2. The apparatus of claim 1 wherein the activated carbon and the potassium permanganate are separated from one another.

3. The apparatus of claim 1, wherein the odorous air passes over the activated carbon before passing over the potassium permanganate, before being recycled.

4. The apparatus of claim 1, wherein the potassium permanganate is supported on a zeolite.

5. The apparatus of claim 1, wherein the filter is disposed in a canister attached to the manifold, and wherein a blower is situated in the canister and the blower is configured to suck air from the toilet bowl through the filter.

6. The apparatus of claim 1, wherein the filter is disposed in a canister located remote from the manifold and connected to the manifold via an intake tube and an outlet tube, and wherein a blower is situated in the canister and the blower is configured to suck air from the toilet bowl through the filter.

7. The apparatus of claim 1, wherein the manifold is integrated into a toilet seat.

8. The apparatus of claim 1, wherein the manifold is separate from the toilet seat.

9. The apparatus of claim 1, wherein a gasket is situated between the manifold and the toilet bowl.

10. The apparatus of claim 1, wherein a gasket is situated between the toilet seat and the toilet lid.

11. The apparatus of claim 7, wherein a gasket is situated between the combined manifold and toilet seat and the toilet lid.

12. The apparatus of claim 1, wherein the potassium permanganate and activated carbon are mixed with one another.

13. The apparatus of claim 1, further comprising a power supply and an on-off switch.

14. The apparatus of claim 1, further comprising an indicator to alert a user of the need to renew the filter material.

15. The apparatus of claim 1, wherein the manifold further comprises an intake port and an exhaust port.

16. The apparatus of claim 15, wherein the intake port is positioned towards the rear of the toilet, and the exhaust port is positioned towards the front of the toilet, thereby ensuring that the flow of air is from the front to the back of the toilet.

17. A method of removing odors from a toilet environment, the method comprising: creating a circuit of closed air from the toilet bowl to an attached manifold; and passing the closed air over a filter in fluid communication with the manifold, wherein the filter comprises activated carbon and potassium permanganate.
